(19) United States
(12) Patent Application Publication
(10) Pub. No.: US 2024/0408376 A1
(43) Pub. Date: Dec. 12, 2024

(54) TUBE ROLLER PUMP

(71) Applicant: B. Braun Avitum AG, Melsungen (DE)

(72) Inventor: Marco Jakobi, Uder (DE)

(21) Appl. No.: 18/735,562

(22) Filed: Jun. 6, 2024

(30) Foreign Application Priority Data
Jun. 7, 2023 (DE) 10 2023 115 053.7

(51) Int. Cl. A61M 60/279; A61M 60/113; A61M 60/37; A61M 60/835; F04B 43/12

(52) U.S. Cl. CPC A61M 60/279; A61M 60/113; A61M 60/37; A61M 60/835; F04B 43/1276; A61M 2205/0238

(57) ABSTRACT

A peristaltic pump for extracorporeal blood treatment includes a pump housing in which a rotor is rotatable about a rotor axis. The rotor has at least two squeeze elements offset in a circumferential direction to each other. The pump housing has a support surface extending arc-shaped about the rotor axis and spaced radially from the rotor. The support surface is configured to support a tube segment which is radially insertable between the rotor and the support surface. In order to extend the service life of the pump and reduce fluctuations in the pump volume, the pump has co-rotating guide surfaces arranged and configured adjacent to both sides of the squeeze element in the circumferential direction in such a way that they each form a circular segment-like guiding passage with the support surface, in which the tube segment can be fixed radially with a predetermined clearance fit.

### CROSS-REFERENCE TO RELATED APPLICATION

This application claims priority under 35 U.S.C. § 119 to German Application No. 10 2023 115 053.7, filed on Jun. 7, 2023, the content of which is incorporated by reference herein in its entirety.

### FIELD

The present disclosure relates to a tube roller pump or peristaltic pump, resp., i.e. a positive displacement pump, in which the medium to be conveyed is forced through a tube by external mechanical deformation of the tube.

### BACKGROUND

Such pumps are frequently used for conveying fluid, in particular blood, in a device for extracorporeal blood treatment, in particular in a dialysis machine. The fluid is conveyed via the peristaltic pump from a low-pressure side to a high-pressure side, wherein an elastically deformable fluid line arranged between the low-pressure side and the high-pressure side in the form of a tube segment, which is referred to as a pump segment, is deformed, in particular squeezed, between a support surface of a pump bed and a rotor with at least two squeeze elements rotating relative thereto.

In a known peristaltic pump, as known for example from the document EP 1 749 549 B1, two squeeze elements 320 diametrically offset from each other in the form of spring-mounted pinch rollers are provided on the rotor 310 with the rotational axis A—as shown schematically in FIGS. 7 to 9—and the support surface 330 is formed by a circular segment area which extends over a sufficiently large centering angle greater than 180°.

In order to fix the tube, i.e. a pump segment 360 (see FIG. 9) inserted in the peristaltic pump, to some extent during operation, there is a guide surface 340 in the middle between the two pinch rollers 320, which is configured like a segment of a circle and extends over a centering angle WZF of approximately 30°. It has been shown that this assembly makes handling more difficult when inserting the pump segment manually.

### SUMMARY

The object of the present disclosure is therefore to further develop a generic peristaltic pump in such a way that handling during manual insertion of the pump segment is simplified.

The present disclosure is based on the realization that if no special measures are taken in order to fix the pump segment in place, the pump segment will move in the pump bed against the conveying direction during operation of the peristaltic pump, causing the pump segment—as shown in FIG. 9—to pull tighter and tighter around the rotor. This can result in damage to the tube and fluctuations in the conveyed volume.

In order to solve the object, co-rotating guide surfaces are provided with the rotor, which are arranged and configured on both sides of the squeeze element, i.e. in pairs adjacent in the circumferential direction, in such a way that they each form a circular segment-like guiding passage with the support surface, in which the tube segment can be fixed radially with a predetermined clearance fit. This results in the additional advantage that not only the conveyed volume of the pump can be kept constant, but also the pump segment is ‘treated’ particularly gently during operation. The curvature characteristics of the tube are defined by the radius of the guide surfaces, which keeps the conveyed volume constant. At the same time, the guide surfaces prevent damage to the pump segment. Since the guide surfaces are only formed in the vicinity of the squeeze elements, the guide contour is divided into several small regions, reducing the contact surface, which also has a positive effect on abrasion on the tube.

If the guide surfaces assigned to a squeeze element and adjacent to it each describe partial segments of a circle, the interaction with a cylindrical or torus-like support surface on both sides of the squeeze element results in circularly curved guiding passages with which the curvature characteristics of the tube can be controlled even better.

If the guide surfaces extend over a total central angle that is dimensioned such that a free space (clearance) extending in the circumferential direction is left between guide surfaces of adjacent squeeze elements, there is the additional advantage that this clearance can be used for manual lateral unlocking of the rotor cover or of the rotor and when inserting the tube or pump segment.

According to an advantageous further development, the guide surfaces assigned to a squeeze element extend over central angles of different sizes. Preferably, the central angle of the guide surface running after/tailing the squeeze element is larger, which allows the tube curvature to be stabilized even better with the lowest possible material consumption.

In principle, the guide surfaces may be attached to any components that move with the rotor. There are particular additional advantages if the guide surfaces are formed on a rotor cover or are optionally attached to it in a replaceable manner. This results in a particularly high degree of design freedom combined with ease of manufacture, since such rotor covers are often configured as injection-molded plastic parts.

In principle, it is possible to use the present disclosure also with peristaltic pumps in which the squeeze elements are at a variable angular distance from each other. However, if the squeeze elements are arranged offset by 180° to each other, it is advantageous to form two guide surfaces that are rotationally symmetrical to each other.

Advantageously, the guide surfaces are made of and/or coated with a material that has abrasion-minimizing sliding properties. It is also advantageous if the surfaces of the guide surfaces have a high surface quality.

### BRIEF DESCRIPTION OF THE DRAWINGS

Exemplary embodiments of the present disclosure are explained in more detail below. The following is shown:

FIG. 1 shows a perspective view of the peristaltic pump with the housing lid open;

FIG. 2 shows a perspective view of a rotor of the peristaltic pump according to FIG. 1;

FIG. 3 shows a top view of the peristaltic pump;

FIG. 4 shows a schematic top view of the peristaltic pump with inserted pump segment;

FIG. 5 shows a schematic top view of the peristaltic pump similar to FIG. 4 with the rotor cover cut free in the area of the squeeze elements;

FIG. 6 shows a top view of the peristaltic pump with the rotor cover removed;

FIG. 7 shows a perspective view of a rotor of a known peristaltic pump;

FIG. 8 shows a schematic top view of the known peristaltic pump with inserted rotor; and

FIG. 9 shows a top view of the known peristaltic pump with inserted pump segment.

### DETAILED DESCRIPTION

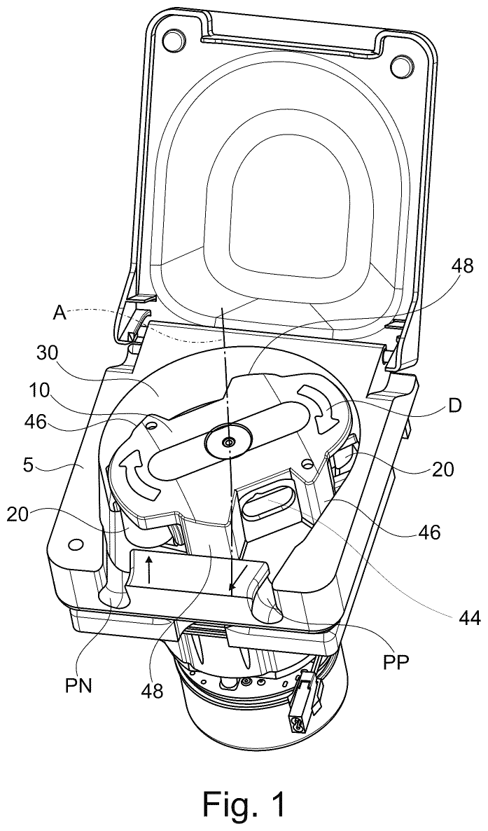

In FIG. 1, a peristaltic pump, as used in dialysis machines, for example, is designated by the reference sign 1. The object of the peristaltic pump is to convey a defined volume of a medium, such as blood or dialysis fluid, by deforming and clamping the elastically deformable fluid line. The peristaltic pump for pumping blood generally pumps from a negative pressure side PN (low-pressure side) to a positive pressure side PP (high-pressure side). The peristaltic pump has a pump housing 5, in which a rotor 10 rotatable about a rotor axis A with at least two squeeze elements 20, here pressure rollers, for example diametrically offset in the circumferential direction to each other, is accommodated and which has a support surface 30 extending arc-shaped about the rotor axis A and being radially spaced from the rotor 20, the support surface 30 being arranged to support a tube segment, not shown in FIG. 1, which can be inserted radially between the rotor 20 and the support surface 30. The support surface 30 is generally formed by a cylinder surface, but it can also be recessed in a trough shape. The rotational direction of the rotor 10 when conveying fluid is indicated by the arrow D.

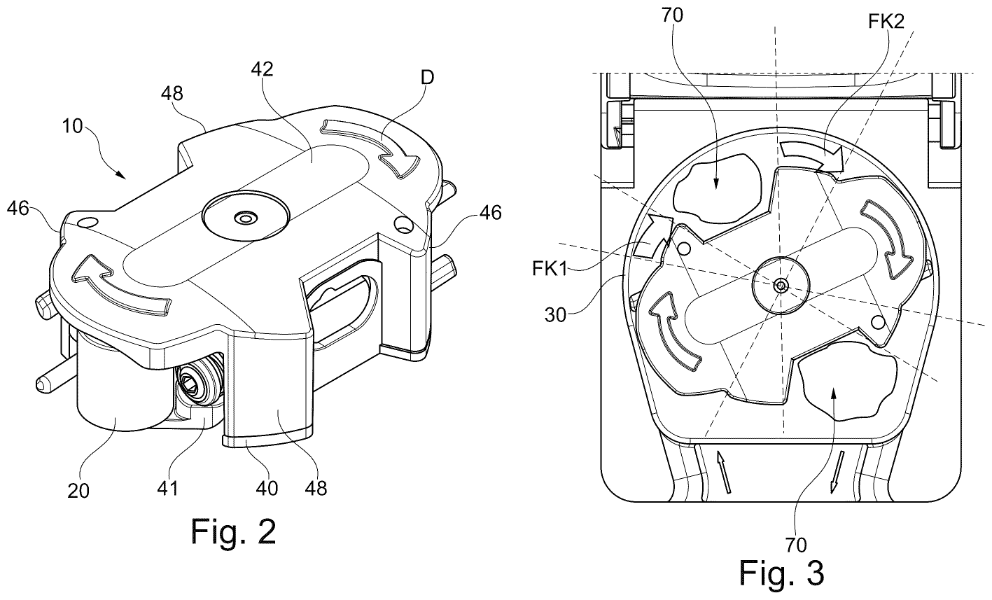

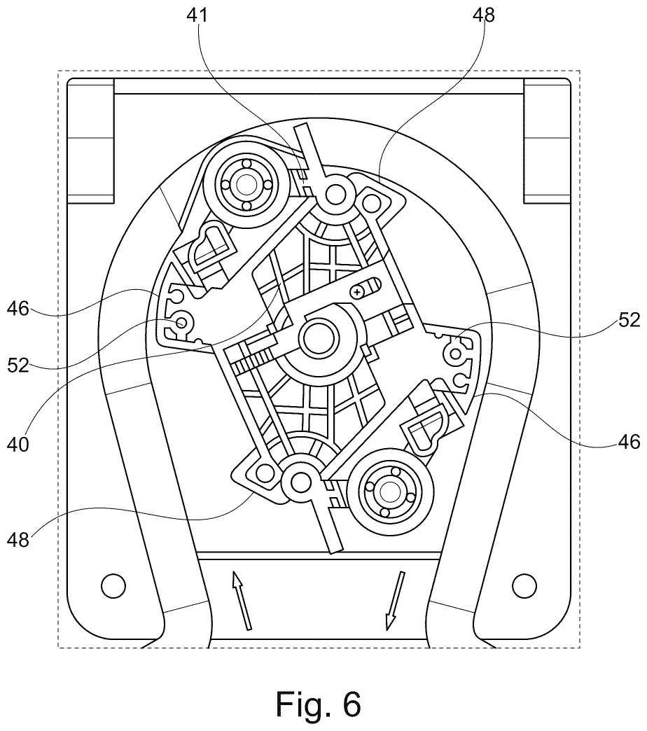

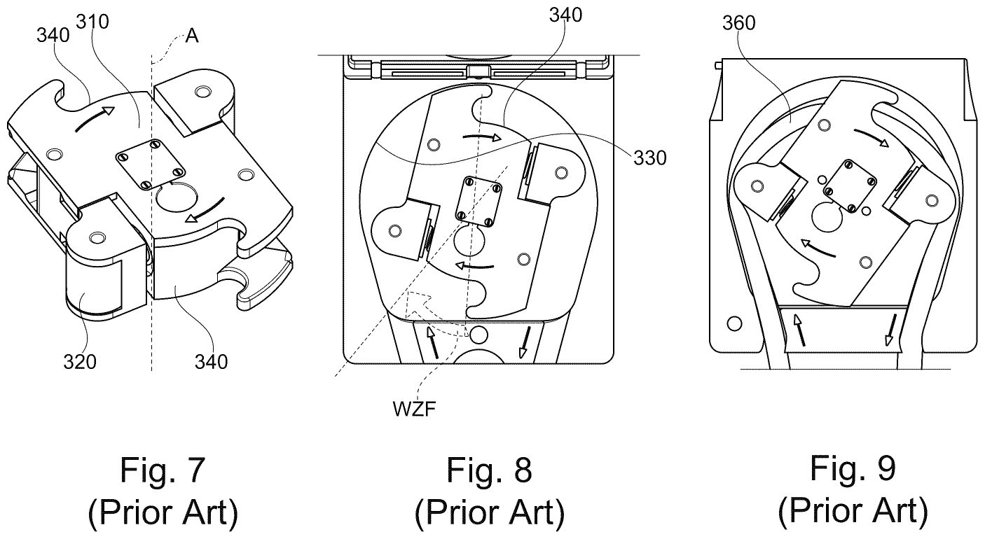

FIG. 2 shows details of the rotor 20. It has a rotor base body 40, visible in plan view in FIG. 6, and a rotor cover 42. The rotor 20 is removable from a drive shaft, which is not shown in detail, via a laterally operable button 44. The squeeze elements 20 are rotatably mounted on a swing arm 41 resiliently supported on the rotor base body 40.

The rotor cover 42 co-rotating with the rotor 10 carries guide surfaces 46, 48 in the area of the squeeze elements 20, of which one guide surface 46 runs before the squeeze element 20 and the other guide surface 48 runs after or trails the squeeze element 20. Accordingly, the guide surfaces 46, 48 are arranged and configured adjacent to both sides of the squeeze element 20 in the circumferential direction in such a way that—as can be seen from FIGS. 3 to 6—they each form a circular segment-like guiding passage FK1 and FK2 with the support surface 30, in which the tube segment 60 inserted into the pump housing 1, which is also referred to as the pump segment, can be fixed radially with a predetermined clearance fit.

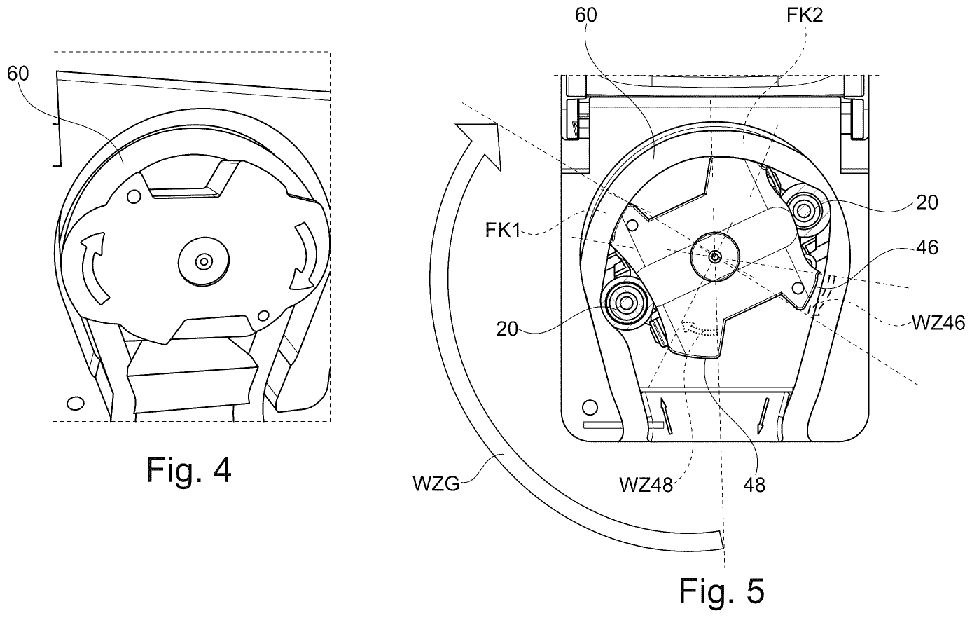

In this way—as can be seen from the representations in FIGS. 4 and 5—the curvature characteristics of the tube 60 during operation of the peristaltic pump are defined via the radius of the guide surfaces. The conveyed volume can thus be kept constant. At the same time, the guide surfaces prevent damage to the pump segment 60. Preferably, the guide surfaces 46, 48 are formed and/or coated from a material that has abrasion-minimizing sliding properties.

As can be seen from the Figures, the configuration or arrangement of the guide surfaces 46, 48 is therefore such that they are only formed in the vicinity of the squeeze elements 20, i.e. at a limited angular distance of approximately 20° to 40° for the leading guide surface 46 and of 35° to 60° for the trailing guide surface 48. In this way, the guide contour is divided into several small regions determining the guiding passages FK1 and FK2, whereby the contact surface is reduced, which also has a positive effect on the abrasion on the tube 60. With this assembly, the guide surfaces 46, 48 extend over a total central angle WZG (see FIG. 5), which is dimensioned, for example with a limitation to 110° to 140°, in such a way that a free space or clearance 70 (see FIG. 3) extending in the circumferential direction is left between adjacent guide surfaces 46 and 48. This clearance 70 can advantageously be used for manual lateral unlocking of the rotor 10 and when inserting the tube or pump segment 60.

In the configuration example shown, the guide surfaces 46, 48 assigned to a squeeze element 20 and adjacent thereto each describe partial segments of a circle. in The interaction with a cylindrical or torus-like support surface 30 between the squeeze elements 20 results—as shown in FIG. 5—in circularly curved guiding passages, with which the curvature characteristics of the tube 60 can be controlled particularly effectively.

For the configuration of the guide surfaces 46, 48, there is a large degree of freedom with regard to position, shape and size. In the configuration example shown, the guide surfaces 46, 48 associated with a squeeze element 20 extend over centering angles of different sizes, with the guide surface 48 running after the squeeze element 20 extending over a larger centering angle WZ48 than the guide surface 46 running before the squeeze element 20 (FIG. 5).

Since the squeeze elements 20 of the peristaltic pump shown are arranged diametrically offset to each other, the leading and trailing guide surfaces 46 and 48 are rotationally symmetrical to each other.

In principle, the guide surfaces 46, 48 may be formed on any component that moves with the rotor 10. In the configuration example shown, they are formed on the rotor cover 42, which is detachably attached to a rotor base body 50. The rotor base body 50 is shown in FIG. 6 in top view with the rotor cover 42 removed. It can be seen that a base 52 of the rotor base body 50 already has the contour of the guide surfaces 46, 48.

Of course, deviations from the configuration example shown are possible without departing from the basic idea of the present disclosure.

For example, the squeeze elements 20 may also be configured to be angularly positionable relative to each other in the direction of rotation.

A component of the peristaltic pump forming the guide surfaces 46, 48 may also be formed in one piece with the rotor base body 40 or may also be replaceably attached to the rotor base body 40 or to the rotor cover 42. The rotor cover 42 may be formed in several parts, for example as a 2-component part, in which case the guide surfaces 46, 48 are formed on inserts.

It lies also within the scope of the present disclosure if the rotor has more than two squeeze elements 20, in particular three or four. The angular positions of the squeeze elements relative to each other, i.e. the angles between adjacent squeeze elements, are preferably 180° outside the range of pre-compression in the case of two squeeze elements, 120° in the case of three squeeze elements, and 90° in the case of four squeeze elements.

The squeeze elements may also be formed directly on the rotor, in particular in one piece with the rotor. Alternatively, they may be arranged on rotor arms. These are preferably configured to pivot in the circumferential direction in relation to the rotor, so that pre-compression can be achieved by pivoting in the circumferential direction. In particular, the squeeze elements may be configured as squeeze rollers or pinch rollers, which roll along the fluid line in a way that is gentle on the material, or sliding shoes, which move smoothly over the fluid line.

The present disclosure thus creates a peristaltic pump, in particular for conveying fluid in a device for extracorporeal blood treatment, with a pump housing in which a rotor rotatable about a rotor axis with at least two squeeze elements offset in a circumferential direction to each other is accommodated and which has a support surface extending arc-shaped about the rotor axis and spaced radially from the rotor, wherein the support surface is configured to support a tube segment which can be inserted radially between the rotor and the support surface. In order to ensure a constant conveyed volume while at the same time protecting the tube segment as far as possible, the peristaltic pump has co-rotating guide surfaces with the rotor, which are arranged and configured adjacent to both sides of the squeeze element in the circumferential direction in such a way that they each form a circular segment-like guiding passage with the support surface. in which the tube segment can be fixed radially with a predetermined clearance fit.

### LIST OF REFERENCE SIGNS

A rotor axis
WZS centering angle of support surface
WZF centering angle of guide surface
PP high-pressure side
PN low-pressure side
D rotational direction
FK1 guiding contour region
FK2 guiding contour region
5 pump housing
10 rotor
20 squeeze element
30 support surface
40 rotor base body
41 swing arm
42 rotor cover
44 button
46 guide surface
WZA6 central angle of 46
48 guide surface
WZA8 central angle of 48
WZG total central angle of 46 and 48
52 basis
60 pump segment
free space (clearance) 70
310 rotor
320 squeeze elements
330 support surface
340 guide surface
5 360 pump segment

1. A peristaltic pump for conveying fluid in a device for extracorporeal blood treatment, the peristaltic pump comprising:
a pump housing that accommodates a rotor that is rotatable inside the pump housing, the rotor being rotatable about a rotor axis and having squeeze elements offset in a circumferential direction to each other, the pump housing having a support surface extending arc-shaped about the rotor axis and spaced radially from the rotor, wherein the support surface is configured to support a tube segment which is radially insertable between the rotor and the support surface; and
guide surfaces that rotate in unison with the rotor, the guide surfaces being configured adjacent to both sides of a squeeze element in the circumferential direction in such a way that the guide surfaces each form a circular segment-like guiding passage with the support surface, in which the tube segment is radially fixable with a predetermined clearance fit.

2. The peristaltic pump according to claim 1, wherein the guide surfaces assigned to a squeeze element and adjacent to the squeeze element each describe partial segments of a circle.

3. The peristaltic pump according to claim 1, wherein the guide surfaces assigned to a squeeze element extend over a total centering angle that is dimensioned such that a free space or clearance extending in the circumferential direction is left between guide surfaces of adjacent squeeze elements.

4. The peristaltic pump according to claim 3, wherein the clearance forms a lateral access for an unlocking button.

5. The peristaltic pump according to claim 1, wherein the guide surfaces assigned to a squeeze element extend over central angles of different sizes.

6. The peristaltic pump according to claim 1, wherein the guide surfaces are formed on a rotor cover.

7. The peristaltic pump according to claim 6, wherein the guide surfaces are attached to the rotor cover in a replaceable manner

8. The peristaltic pump according to claim 1, wherein the squeeze elements are arranged diametrically offset to each other in the peristaltic pump.

9. The peristaltic pump according to claim 8, wherein the guide surfaces are each configured to be rotationally symmetrical to each other.

10. The peristaltic pump according to claim 1, wherein the guide surfaces are made of a material that has abrasion-minimizing sliding properties.

11. The peristaltic pump according to claim 1, wherein the guide surfaces are coated with a material that has abrasion-minimizing sliding properties.
